(19) United States
(12) Patent Application Publication
(10) Pub. No.: US 2017/0215700 A1
(43) Pub. Date: Aug. 3, 2017

(54) PORTABLE MEDICAL PLATFORM SYSTEM OF AN ENDOSCOPE

(71) Applicant: CHIYI TECHNOLOGY CO., LTD., New Taipei City (TW)

(72) Inventor: YEN TSUNG LIN, NEW TAIPEI CITY (TW)

(21) Appl. No.: 15/014,120

(22) Filed: Feb. 3, 2016

(51) Int. Cl. A61B 1/00; A61B 1/06

(52) U.S. Cl. CPC A61B 1/00066; A61B 1/0676; A61B 1/00022; A61B 1/0684

(57) ABSTRACT

The present invention relates to a portable medical platform system of an endoscope, which includes a camera head, a tube, a handle, and a portable main system. The camera head having light sources, a sensor, and an image signal processor is mounted in a distal end of the tube. The other proximal end of the tube is connected with the handle which has provided with several functional buttons thereon for electrically connecting with a control board to control actions of light sources and the camera head wherein the portable main system is also connected with the control board. By providing the control system on the handle with separated control board, the invention therefore obtains utility and improvement for a medical surgery.

### FIELD OF THE INVENTION

The present invention relates to a portable medical platform system of an endoscope, which provides a control board in a handle to control functions of a camera head including a sensor, an image signal processor, and light sources, and the control board is separated from the main system that is facilitate to be used on the portable platform system and therefore obtains utility for going and monitoring a medical surgery.

### BACKGROUND OF THE INVENTION

A conventional endoscope usually includes a main system and a tube with a camera head assembly. Most control parts are provided on the main system, and the volume of the main system is therefore very large. The known main system also includes several independent machines to control different functions of the endoscope. Hence, the prior system cannot be portable and is inconvenient as proceeding medical surgeries.

A Taiwan patent application no. 103141282 as well as U.S. patent Ser. No. 14/953,267 is related to improve quality of transport image that is still lack of facilitating for medical proceeding. Moreover, the connecting elements of each mechanism are specifically unique without commonality that causes high expense of fixed cost and is lack of economic competition for that the elements of the known endoscope are always used repeatedly. If the elements are not completely sterilized, it may cause risks of infection that should be improved. As shown in FIG. 1, it is an endoscope system block diagram developed by TI corp. which focuses in providing an entire image chain and LED drivers for better directionality and less waste heat. Depending on the image sensor's location, low-noise line drivers may be needed to transmit the signal over the light tube's length. Critical considerations for line drivers are low power, noise immunity and data rate. And the final image quality is the analog front end (AFE), and so on. This still concerns how to get a better image but not about facilitating for medical proceeding.

### SUMMARY OF THE INVENTION

The present invention relates to a portable medical platform system of an endoscope, which includes a camera head, a tube, a handle, and a portable system. The camera head connected with a distal end of the tube has a sensor, an image signal process, and light sources, each of which is electrically connected to a control board in the handle. A proximal end of the tube in connected with the handle having several buttons thereon to control functions of lighting and picture taking of the camera head. The portable system is connected to the control board in the handle. Hence, the doctor or other medical personnel can easily hold the handle to operate a medical surgery and keep going or monitoring the medical treatment more conveniently.

Now, accompanying with drawings, the character and structure of the present invention will be disclosed as following.

### BRIEF DESCRIPTION OF THE DRAWINGS

FIG. 1 is a known system block diagram of an endoscope.

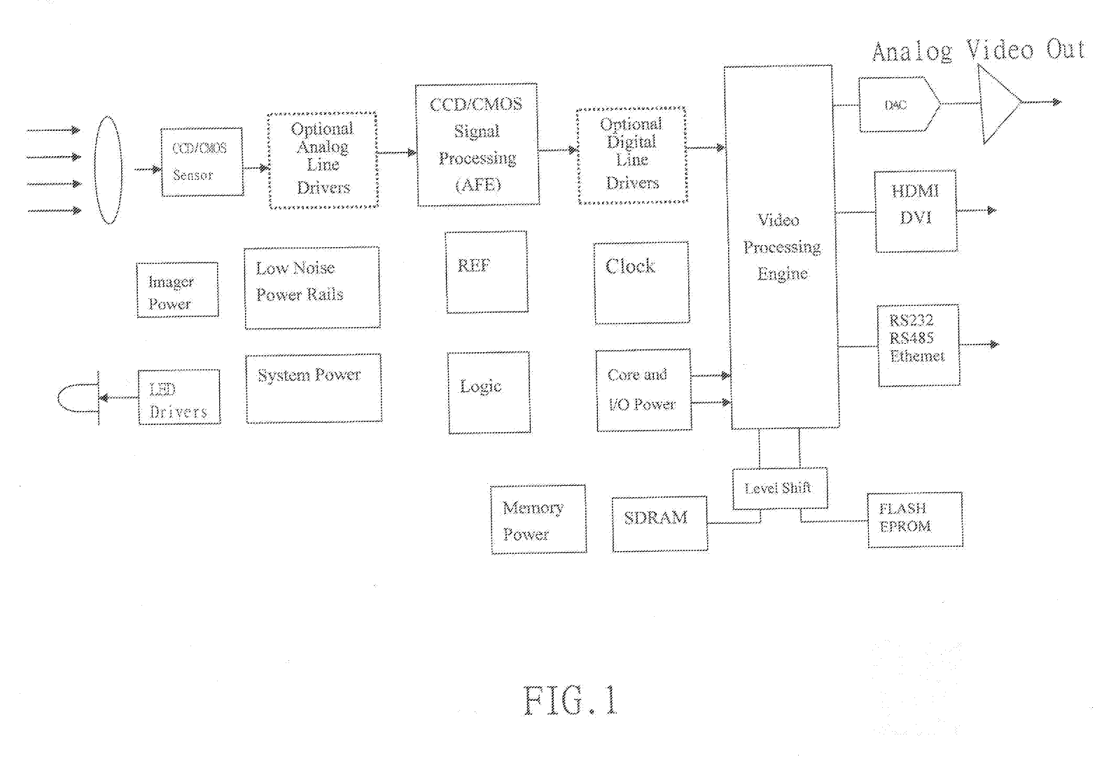

FIG. 2 is a system block diagram of an endoscope according to the present invention.

FIG. 3 is a modified system block diagram of an endoscope according to the present invention.

### DETAILED DESCRIPTION OF THE PREFERRED EMBODIMENT

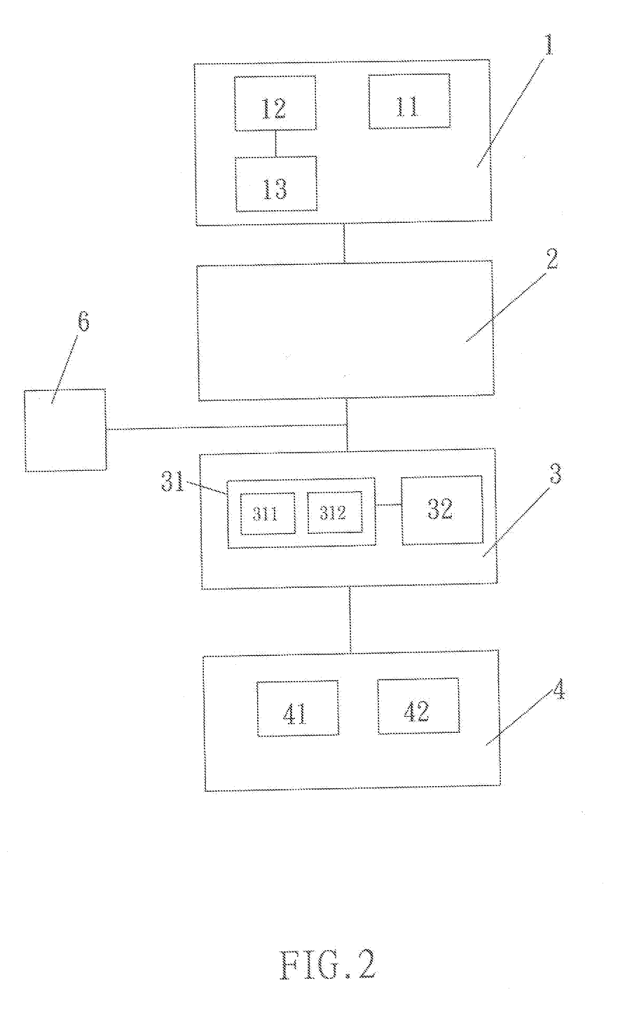

Please referring to FIG. 2, the present invention relates to a portable platform system of an endoscope including a camera head (1), a tube (2), a handle (3), and a portable system (4). The tube (2) has its distal end connecting with the camera head (1) and its proximal end with the handle (3), while the main system (4) is connected to the handle (3) also. The said camera head (1) includes at least light sources (11), a sensor (12), and an image signal processor (13). The light sources (11) and the image signal processor (13) are electrically connected to a control board (31) in the handle (3) by electrical wires that pass through the tube (2). The handle (3) is provided with several buttons (32) thereon, which are electrically connecting respectively to a light control mechanism (311) and an image-taking controlling mechanism (312) mounted on the control board (31) to operate the functions of the light sources (11) and the camera head (1). The portable system (4) is also electrically connected to the control board (31) in the handle (3), which can connect with a monitor (41) for receiving the signals obtained from the camera head (1) directly, and can be provided with memory storage bus (42) for recording all data during the medical treatment.

In above embodiment, the light sources (11) can be composed by several LEDs which are capable of providing with various wavelength of light. It can be operated by controlling one of the buttons on the handle (3). Another button can be selected to control the image signal processor (13) through the control board (31). Accordingly, the medical personnel can just use his one hand holding the handle (3) to proceed a medical surgery easily, while he or she can immediately watch the image of internal organs from the monitor (41) on the portable system (4) that will facilitate the medical treatment obviously.

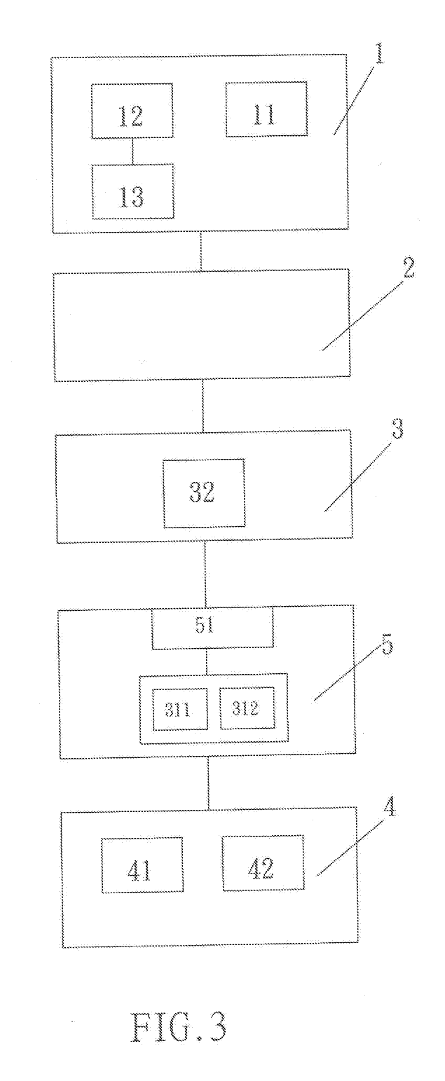

Please refer to FIG. 3, it shows a modified embodiment of the present invention. The characteristic of this is to provide the control board (31) separately located beside the handle (3) as an independent mini board (5). The electrical wires connecting with the light sources (11) and the image signal processor (13) of the camera head (1) as well as the function buttons (32) are extended through the handle (3) and combined with a USB connector (51) for connecting with the control board (31) in the mini board (5). This assembly can be operated as aforesaid conveniently. And at this embodiment, since the handle (3), the tube (2), and the camera head (1) can be separated from the mini board (5) easily, they can be sterilized simply to keep from contamination or can be deposable directly because of their low cost that becomes a one-time-uses parts to ensure preventing from medical treatment infection.

Furthermore, the present invention can also be provided with a TV out connecting port from the image signal processor (13) to transmit the live image of the medical surgery on a connected TV (6), from which other monitoring persons can watch the medical is treatment at the same time to assist the person who is doing the surgery.

Therefore, the present invention obviously obtains improvement and facilitates. And it is to be understood that any modified design according to the merit of the present invention will be still claimed in this application.

I claim:

1. A portable platform system of an endoscope including a camera head, a tube, a handle, and a portable system, wherein the tube has its distal end connecting with the camera head and its proximal end with the handle, while the main system is connected to the handle also; the said camera head including at least light sources, a sensor, and an image signal processor, in which the light sources and the image signal processor are electrically connected to a control board in the handle by electrical wires that pass through the tube; the handle being provided with several buttons thereon, which are electrically connecting respectively to the control board to operate the functions of the light sources and the camera head; and the portable system being also electrically connected to the control board in the handle, which can connect with a monitor for receiving the signals obtained from the camera head directly.

2. The portable platform system of an endoscope as claimed in claim 1, wherein the control board includes a light control mechanism and an image-taking controlling mechanism.

3. The portable platform system of an endoscope as claimed in claim 1, wherein the portable system includes a USB memory bus for recording all image obtained during the medical surgery.

4. The portable platform system of an endoscope as claimed in claim 1, wherein the light sources includes several LEDs, which has various wavelength.

5. A portable platform system of an endoscope including a camera head, a tube, a handle, and a portable system, wherein the tube has its distal end connecting with the camera head and its proximal end with the handle, while the main system is connected to the handle also; the said camera head including at least light sources, a sensor, and an image signal processor, in which the light sources and the image signal processor are electrically connected to a mini board provided beside the handle by electrical wires that pass through the tube and the handle; the handle being provided with several buttons thereon, which are electrically connecting respectively by electrical wires extended to the mini board to operate the functions of the light sources and the camera head; and the portable system being also electrically connected to the mini board, which can connect with a monitor for receiving the image signals obtained from the camera head directly.

6. The portable platform system of an endoscope as claimed in claim 2, wherein the mini board includes a light control mechanism and an image-taking controlling mechanism.

7. The portable platform system of an endoscope as claimed in claim 2, wherein the portable system includes a USB memory bus for recording all image obtained during the medical surgery.

8. The portable platform system of an endoscope as claimed in claim 2, wherein the light sources includes several LEDs, which has various wavelength.
